(19) United States
(12) Patent Application Publication
(10) Pub. No.: US 2025/0241735 A1
(43) Pub. Date: Jul. 31, 2025

(54) SYSTEM AND METHOD FOR CERAMIC INKJET MESH PRINTING

(71) Applicant: Zima International, Inc. d/b/a Dandy, New York, NY (US)

(72) Inventors: Srinivasan Sundararajan, New York, NY (US); Kevin Myers, New York, NY (US); Adrian Looi, New York, NY (US); Kenn Butler, New York, NY (US); Daniel Hanover, New York, NY (US); Oluwatoniloba Oloko, New York, NY (US)

(21) Appl. No.: 19/041,426

(22) Filed: Jan. 30, 2025

Related U.S. Application Data
(60) Provisional application No. 63/627,670, filed on Jan. 31, 2024.

(51) Int. Cl. A61C 13/08; A61C 13/083; A61C 13/34; B41J 3/407; B41J 29/00; B41M 5/00

(52) U.S. Cl. CPC A61C 13/082; A61C 13/083; A61C 13/34; B41J 3/4073; B41J 29/00; B41M 5/0088; A61C 2201/002

(57) ABSTRACT

Disclosed are systems and methods for a computerized framework for automated ceramic inkjet printing in dental restoration manufacturing. The disclosed ceramic inkjet mesh printing for dental restorations combines precision digital control with ceramic materials to create highly accurate and aesthetically pleasing dental prosthetics. The disclosed mechanisms utilize specialized printheads designed to deposit precise amounts of ceramic materials onto dental substrates, enabling the creation of detailed color gradients and natural-looking surfaces that closely mimic natural teeth.

### CROSS-REFERENCE TO RELATED APPLICATIONS

The instant application claims the benefit of priority from U.S. Provisional Patent Application No. 63/627,670, filed Jan. 31, 2024, which is incorporated herein by reference in its entirety.

### FIELD OF THE DISCLOSURE

The present disclosure is generally related to dental restoration, and more particularly, to systems and methods for implementing ceramic mesh printing robotics for dental restorations.

### SUMMARY OF THE DISCLOSURE

According to some embodiments, as discussed herein, disclosed are systems and methods for a computerized framework for automated ceramic inkjet printing in dental restoration manufacturing. As provided herein, by way of example according to some embodiments, the disclosed systems and methods function for an inkjet or nozzle to deposit a curated slurry which contains a ceramic and pigment onto surfaces of a dental restoration.

According to some embodiments, as discussed herein, ceramic inkjet mesh printing for dental restorations represents a significant advancement in dental manufacturing technology, combining precision digital control with traditional ceramic materials to create highly accurate and aesthetically pleasing dental prosthetics. The disclosed framework utilizes specialized printheads designed to deposit precise amounts of ceramic materials onto dental substrates, enabling the creation of detailed color gradients and natural-looking surfaces that closely mimic natural teeth.

According to some embodiments, as discussed in more detail below, the framework can operate by preparing a ceramic restoration substrate, which undergoes precise three-dimensional (3D) scanning to establish spatial coordinates and surface mapping. Such digital mapping ensures accurate material deposition across the restoration's complex geometries, including challenging areas such as occlusal surfaces and margin regions. The disclosed printing system, discussed infra, employs multiple printheads, which for example, can be configured with separate heads for stain pigments and glazing materials, allowing for precise control over color application and surface finishing.

In some embodiments, the framework can implement material formulation with specialized ceramic inks developed to achieve optimal viscosity and surface tension characteristics. Such inks, for example, can include a combination of glass-based glazes and color pigments, with ratios approximately 30% glaze to 70% pigment for staining applications. As discussed herein, the disclosed processing can maintain automated control over throw distance, typically optimized at around five millimeters between printhead and substrate surface, ensuring consistent drop placement and pattern formation.

In some embodiments, the disclosed printing framework can incorporate thermal management mechanisms, which can utilize preheating mechanisms to optimize substrate surface conditions for ink adhesion while carefully avoiding premature ink drying at the printheads. Such thermal control, combined with precise n-axis (e.g., five-axis) positioning capabilities, enables the system to maintain consistent application quality across varying surface geometries and orientations. The disclosed processing can involve a set or predetermined number of passes, with initial passes focusing on color application followed by additional passes for stain and glaze buildup to achieve desired thickness and surface characteristics.

According to some embodiments, the disclosed framework can perform quality control operations via real-time (or near-real time, or criteria/event-based) monitoring of multiple parameters, including, but not limited to, spatial positioning, temperature conditions, material deposition patterns and the like, or some combination thereof. As provided herein, the disclosed processing can achieve threshold satisfying levels of accuracy in color matching and surface finish, with cycle times typically ranging from 90 to 120 seconds per restoration, though ongoing developments aim to optimize these processing times through improvements in movement efficiency, thermal processing, and material formulation.

Accordingly, the disclosed manufacturing mechanisms and operations provide several advantages over traditional dental restoration coloring methods, including improved consistency, reduced processing times, and the ability to achieve complex color gradients and patterns that closely match natural dentition. The disclosed systems and methods can provide improvements in optimizing material formulations and enhancing processing efficiency while maintaining the high quality standards required for dental applications.

According to some embodiments, a method is disclosed for ceramic mesh printing robotics to perform robotic inkjet staining for dental restorations. In accordance with some embodiments, the present disclosure provides a non-transitory computer-readable storage medium for carrying out the above-mentioned technical steps of the framework's functionality. The non-transitory computer-readable storage medium has tangibly stored thereon, or tangibly encoded thereon, computer readable instructions that when executed by a device cause at least one processor to perform ceramic mesh printing robotics for inkjet staining for dental restorations.

In accordance with one or more embodiments, a system is provided that includes one or more processors and/or computing devices configured to provide functionality in accordance with such embodiments. In accordance with one or more embodiments, functionality is embodied in steps of a method performed by at least one computing device. In accordance with one or more embodiments, program code (or program logic) executed by a processor(s) of a computing device to implement functionality in accordance with one or more such embodiments is embodied in, by and/or on a non-transitory computer-readable medium.

### DESCRIPTIONS OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a perspective view of a system for applying ceramic glaze to a dental restoration using an inkjet printer head according to some embodiments of the present disclosure;

FIG. 2A is a side view of the system of FIG. 1 in a first position according to some embodiments of the present disclosure;

FIGS. 2B and 2C provide non-limiting example views of the system of FIG. 1 according to some embodiments of the present disclosure;

FIG. 2D provides a non-limiting example view of the system of FIG. 1 according to some embodiments of the present disclosure;

FIG. 3 is a side view of the system of FIG. 1 in a second position according to some embodiments of the present disclosure;

FIG. 4A is a perspective view of the dental restoration and the inkjet printer head of FIG. 1 in a first orientation according to some embodiments of the present disclosure;

FIG. 4B is a side view of the dental restoration and the inkjet printer head of FIG. 1 in a second orientation according to some embodiments of the present disclosure;

FIG. 4C is a side view of the dental restoration and the inkjet printer head of FIG. 1 in a third orientation according to some embodiments of the present disclosure;

FIG. 4D is a side view of the dental restoration and the inkjet printer head of FIG. 1 in a fourth orientation according to some embodiments of the present disclosure;

FIG. 5 depicts example views of a restoration component under a scanner according to some embodiments of the present disclosure;

FIG. 6 is a block diagram of an example configuration within which the systems and methods disclosed herein could be implemented according to some embodiments of the present disclosure;

FIG. 7 is a block diagram illustrating components of an exemplary system according to some embodiments of the present disclosure; and

FIG. 8 illustrates an exemplary workflow according to some embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or user, entity, subscriber or customer) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

A client device may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

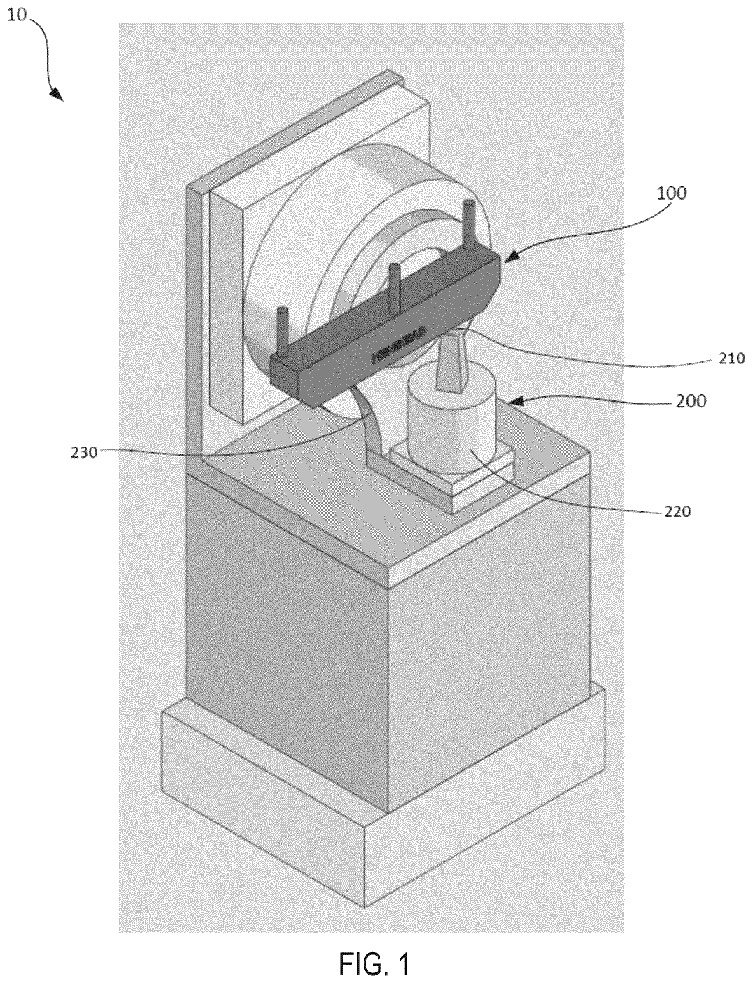

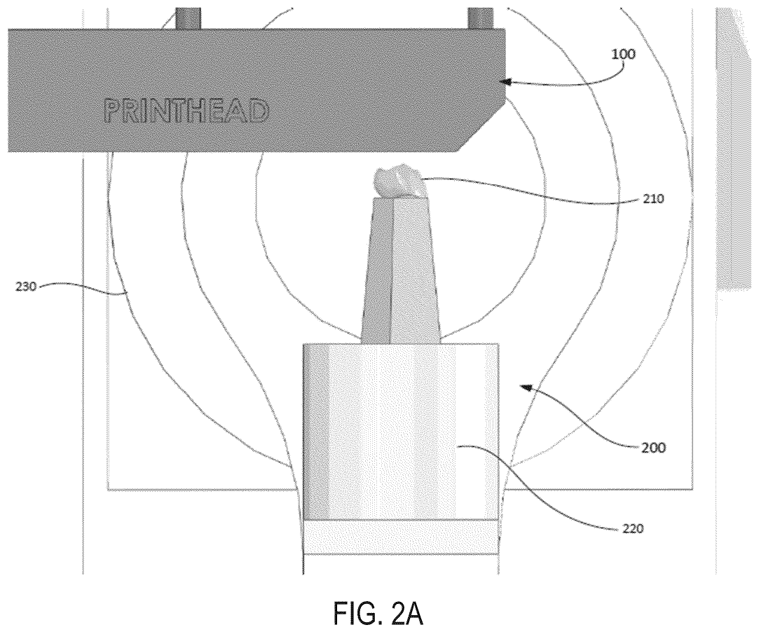

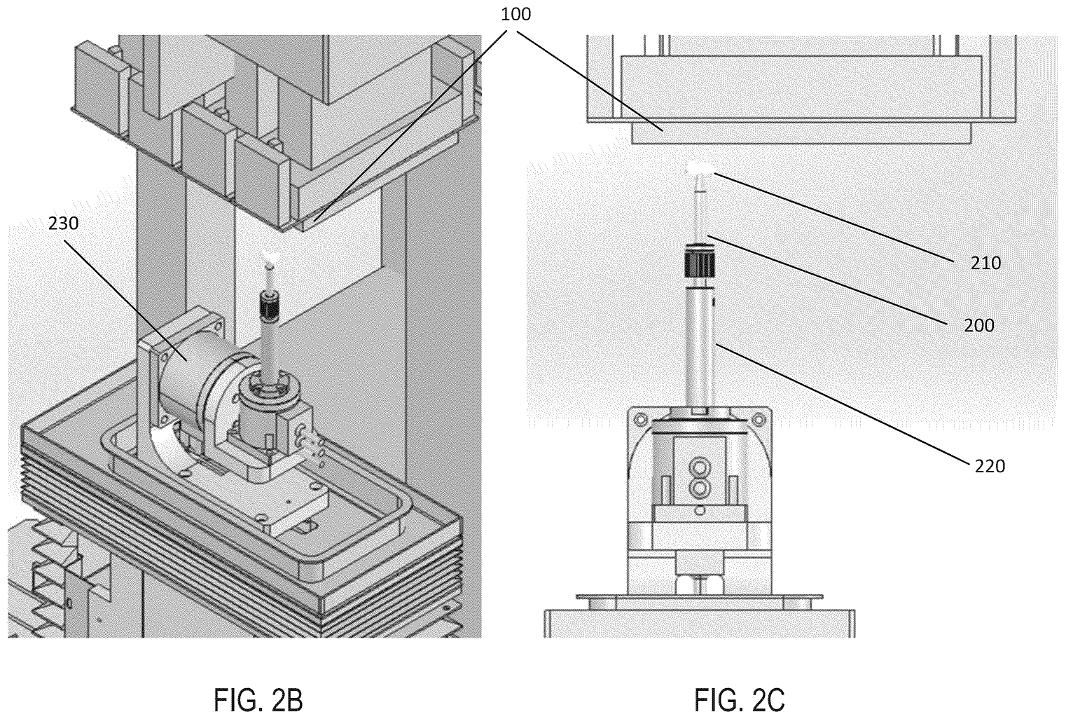

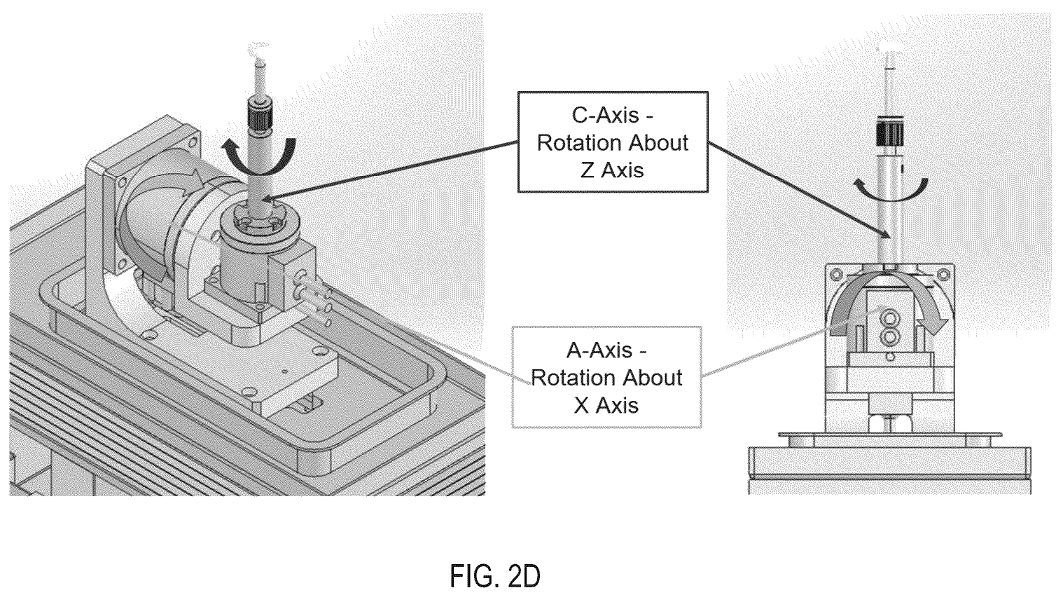

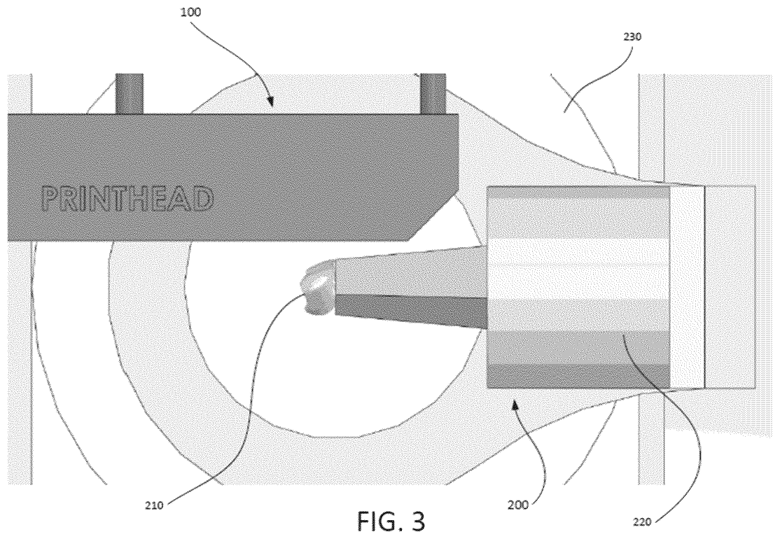

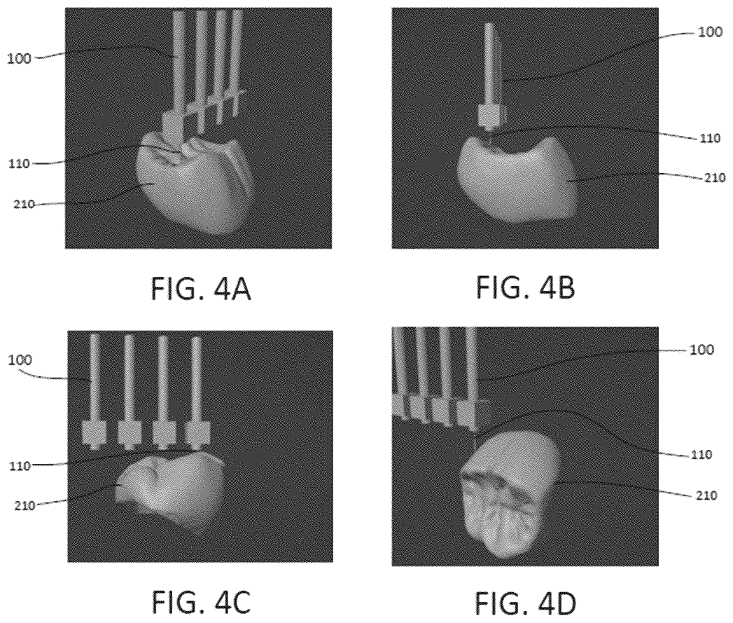

Certain embodiments and principles will be discussed in more detail with reference to the figures. As shown in FIG. 1, a system 10 for applying a ceramic glaze to a dental restoration 210 using an inkjet printer is shown to include an inkjet printer head 100 and a restoration holder 200. The restoration holder 200 is shown to include a restoration 210, a base 220, and a rotation mechanism 230. In some embodiments (including the embodiment shown), the inkjet printer head 100 has a fixed position, such that the location of the glaze application (e.g., droplet stream 110) from the inkjet printer head 100 has a fixed origin. In these embodiments, the different sides of the restoration 210 are exposed to the droplet stream via movement of the restoration 210 itself, which may be accomplished via movement of the base 220 about a Y-axis (relative to the views of FIGS. 2A-2D and 3) and/or the rotation mechanism 230 about a Z-axis (relative to the views of FIGS. 2A-2D and 3). FIGS. 4A-D illustrate various positions of the restoration 210 relative to the inkjet printer head 100. In other embodiments, the inkjet printer head 100 may be capable of movement while the restoration 210 is stationary, such that the inkjet printer head 100 moves about the restoration 210 in order to apply glaze to each portion of the restoration 210, as needed. In further embodiments, both the inkjet printer head 100 and the restoration 210 are movable, such that each portion of the restoration 210 is glazed through a combination of both components moving.

The movement capability of the restoration holder 200 is illustrated in FIGS. 2A-2D and 3. FIG. 2A shows the restoration holder 200 in a first position in which the base 220 is positioned at 6 o'clock relative to a “clock” defined by the rotation mechanism 230. FIG. 3 shows the restoration holder 200 in a second position in which the base 220 is positioned at 3 o'clock relative to the “clock” defined by the rotation mechanism 230. The transition from the first position of FIG. 2A to the second position of FIG. 3 is accomplished via rotation of the rotation mechanism 230. Because the rotation mechanism 230 is shaped such that the restoration 210 is substantially in the center of the rotation mechanism 230, a distance between the inkjet printer head 100 and the restoration 210 remains substantially the same throughout rotation of the rotation mechanism 230. Similarly, the restoration 210 is positioned in the relative center of the base 220, so the rotation of the base 220 does not affect the distance between the restoration 210 and the inkjet printer head 100. Maintaining a consistent distance between the restoration 210 and the inkjet printer head 100 is important to the functionality of the system 10, as inconsistencies can cascade into inconsistent coloration.

As depicted in FIGS. 2B and 2C, depicted are additional views of non-limiting example embodiments of the depictions in FIG. 2A. FIG. 2B depicts a view of the printhead 100 and rotational mechanism 230; and FIG. 2C depicts a view of the first position as provided in FIG. 2A, discussed supra.

In FIGS. 2E, provided is a non-limiting embodiment of functionality of the rotational mechanism 230, as discussed above respective to FIG. 2A and FIG. 3, and in more detail below.

Accordingly, such disclosure in FIGS. 2A-2D and FIG. 3 provide non-limiting printing methodologies for which dynamic movement of a restoration under a printer head 100 (or heads) can be utilized/implemented.

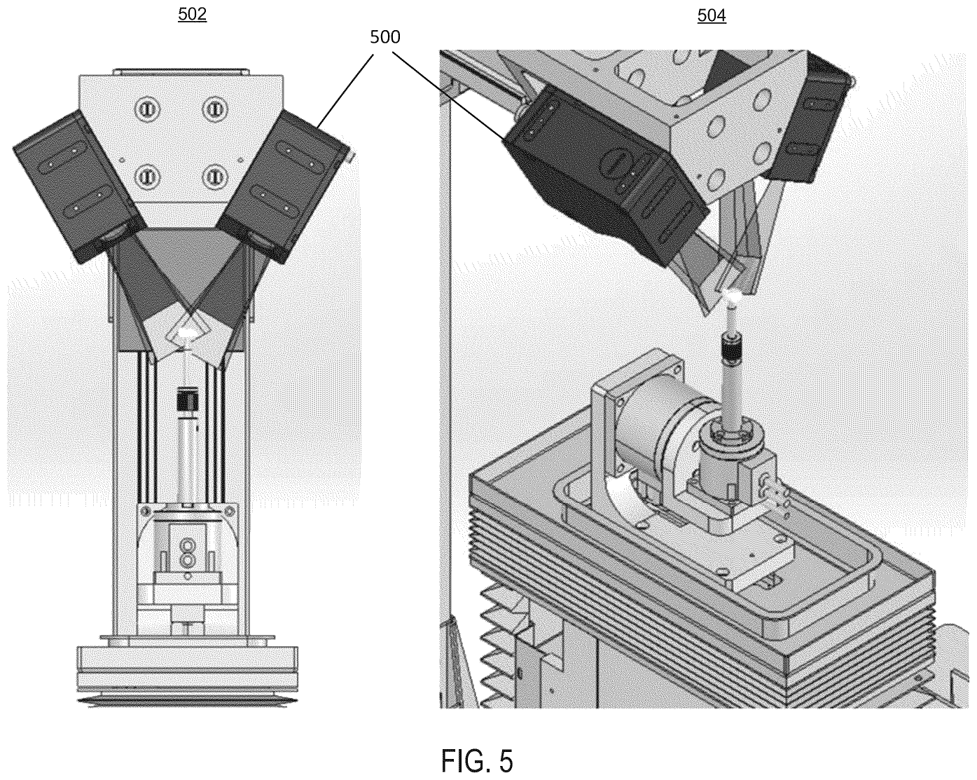

Turning to FIG. 5, depicted is a non-limiting example embodiment of restoration 210 being scanned by a scanner 500. Example view 502 and view 504 provide example depicts of a scanner being utilized to capture information related to a dental restoration component. That is, according to some embodiments, in order to determine the coloration pattern to be printed, the system 10 may utilize a scanner (e.g., an optical scanner) to generate a scan of a real tooth (or teeth) of the patient. The scan may be a 3D rendering of the scanned tooth (or teeth) and may be analyzed in order to derive a coloration pattern for printing. In some embodiments, the scan may be taken of the teeth that are positioned on either side of the position for the dental restoration (e.g., the gap into which the dental restoration will be fixed), such that the coloration pattern is derived based on those teeth closest to the dental restoration in order to maximize the aesthetic consistency of the dental restoration.

The derived coloration pattern may be a 2D map or plot that defines a number of points and a color profile (e.g., tone, hue, base color, and the like) that corresponds to each point. The 2D map may then be virtually applied to a 3D rendering of the dental restoration (e.g., like a vinyl wrap for a vehicle), with adjustments made—either automatically or via human input—in order to determine a coloration profile for the entire dental restoration. In some embodiments—such as those in which the dental restoration is a near-exact replica of another tooth (e.g., the dental restoration for a right incisor is based on the left incisor), the coloration profile may be derived directly from the other tooth.

Once the 3D coloration profile is established, the system 10 may determine a rotation profile for the restoration holder 200 and a print pattern for the inkjet printer head 100. The rotation profile may include commands for moving the base 220 and the rotation mechanism 230 that, in coordination with the print pattern, ensure that each portion of the dental restoration 210 is exposed to ceramic ink. The print pattern may include commands for dispensing ceramic ink via the inkjet printer head 100 at commanded quantities, qualities (e.g., color combinations), and timings of ink. In particular, the print pattern may account for changes in topography across the surface of the dental restoration by affecting the speed and/or volume of a particular droplet stream. Both the rotation profile and the print pattern may be optimized in order to reduce the overall time spent applying glaze.

This system overcomes several notable technical issues. First, the size (about 10 microns) of the droplets of the manually-applied ceramic ink exceeds the capability (<1 micron) of most inkjet printing heads. In some embodiments, the system makes use of a specially-designed inkjet head that enables passage for the larger droplets. In some embodiments, a different formulation for the ceramic ink is developed that performs a similar function to the original ceramic ink but at a reduced size capable of use with standard inkjet heads.

Second, the cyan-yellow-magenta-white (CYMW) color palette that is standard for inkjet printing may not satisfactorily match the various shades of multichromatic real teeth. In some embodiments, the system utilizes a bespoke collection of ink colors that, either alone or in combination, is capable of matching any required coloration pattern.

Third, the applied ceramic glaze must also match the translucency (e.g., reflectivity) of a real tooth, in addition to the coloration pattern. To this end, the system may apply various amounts of clear and/or blue (which appears translucent as ceramic) ink in addition to the quantities of ink applied for the specific coloration pattern. Because these translucent layers are different from the color layers, the system accounts for both when determining the printing pattern, as a too-thick layer of glaze can impact the fit, appearance, or performance of the dental restoration.

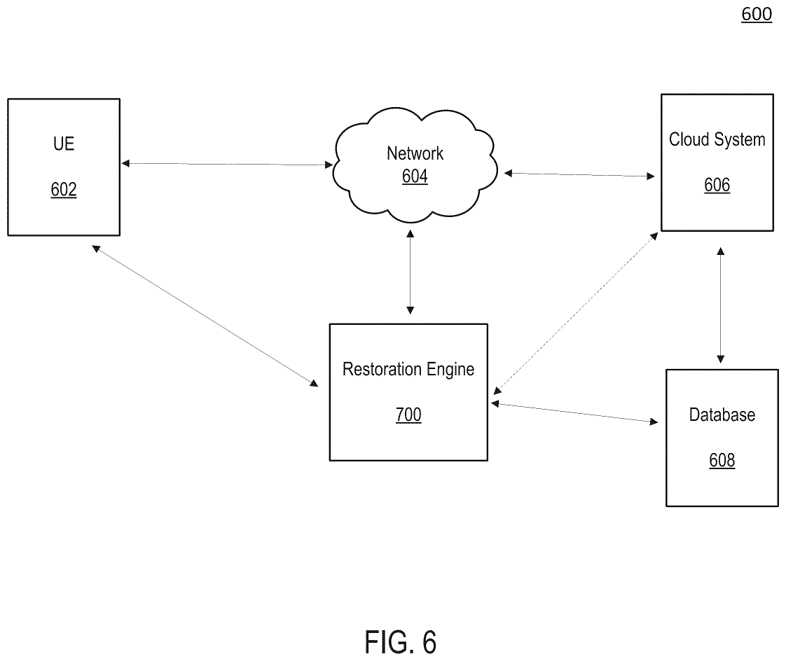

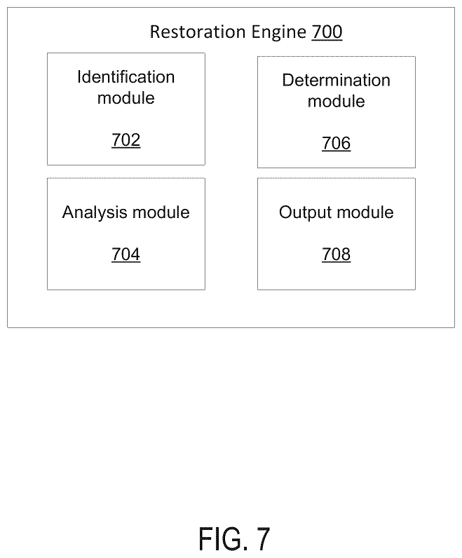

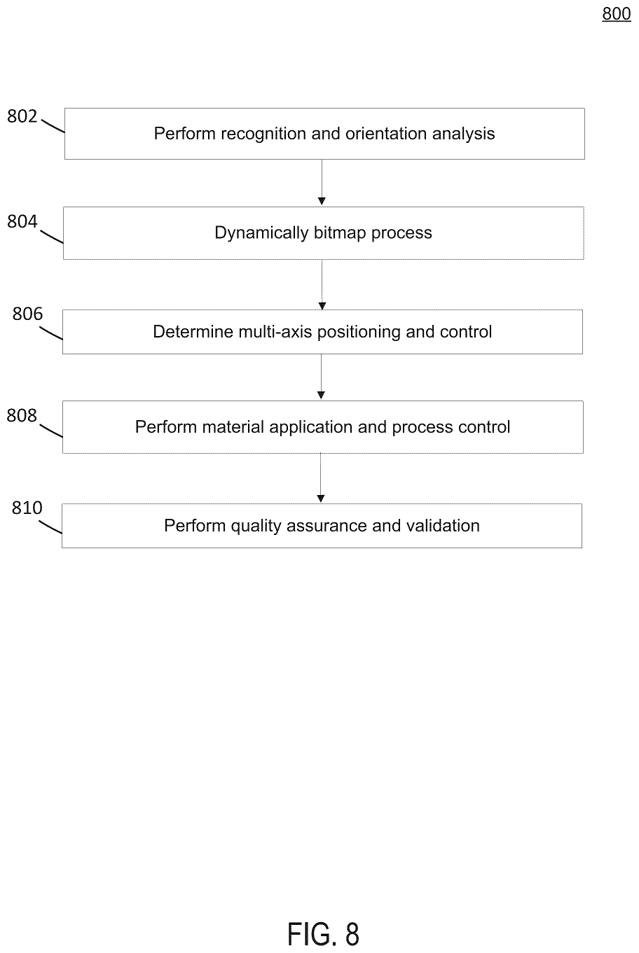

Turning to FIGS. 6-8, disclosed are non-limiting embodiments for implementing the above functional components of the disclosed system 10 as provided via FIGS. 1-5. Accordingly, FIG. 6 provides an example operating environment for which such system(s) can function; FIG. 7 provides engine 700 which represents the computerized framework for providing the novel color matching and glazing, as discussed herein; and FIG. 8 provides Process 800 which details non-limiting example embodiments for execution of engine 700 within a runtime environment.

According to some embodiments, the disclosed systems and methods combine 3D scanning, spatial analysis, and precision inkjet printing capabilities. The instant disclosure provides a novel framework for ceramic inkjet printing in dental restoration manufacturing, focusing on its operational methodology, technical capabilities, and process optimization strategies, as provided below in more detail. Accordingly, the disclosed framework provides improved capabilities for managing complex dental restorations via integration of multiple processing technologies and control systems, such that consistent, high-quality results can be achieved.

With reference to FIG. 6, a system 600, similar to system 10, discussed supra, is depicted which includes user equipment (UE) 602, network 604, cloud system 606, database 608 and restoration engine 700. It should be understood that while system 600 is depicted as including such components, it should not be construed as limiting, as one of ordinary skill in the art would readily understand that varying numbers of UEs, cloud systems, databases and networks, inter alia, can be utilized; however, for purposes of explanation, system 600 is discussed in relation to the example depiction in FIG. 6.

According to some embodiments, UE 602 can represent the printer system (e.g., system 10 and/or the printer from FIGS. 1-5, discussed supra). In some embodiments, UE 602 can correspond to any other device that can connect to system 10 and control its operation, such as, for example, a mobile phone, tablet, laptop, sensor, Internet of Things (IoT) device, autonomous machine, and any other device equipped with a cellular or wireless or wired transceiver.

In some embodiments, a peripheral device (not shown) can be connected to UE 602, and can be any type of peripheral device, such as, but not limited to, a wearable device (e.g., smart watch), printer, display, speaker, sensor, and the like. In some embodiments, a peripheral device can be a 3D scanner or scanning device, and in some embodiments, UE 602 can be the scanning device, as discussed herein.

In some embodiments, a peripheral device can be any type of device that is connectable to UE 602 via any type of known or to be known pairing mechanism, including, but not limited to, Wi-Fi, Bluetooth™, Bluetooth Low Energy (BLE), NFC, and the like.

In some embodiments, network 604 can be any type of network, such as, but not limited to, a wireless network, cellular network, the Internet, and the like (as discussed above). Network 604 facilitates connectivity of the components of system 600, as illustrated in FIG. 6.

According to some embodiments, cloud system 606 may be any type of cloud operating platform and/or network based system upon which applications, operations, and/or other forms of network resources may be located. For example, system 606 may be a content provider, service provider and/or network provider from where services and/or applications may be accessed, sourced or executed from. For example, system 606 can represent the cloud-based architecture associated with a system provider (e.g., healthcare company, private company, and the like, for example), which has associated network resources hosted on the internet or private network (e.g., network 604), which enables (via engine 700) the dental management discussed herein.

In some embodiments, cloud system 606 may include a server(s) and/or a database of information which is accessible over network 604. In some embodiments, a database 608 of cloud system 606 may store a dataset of data and metadata associated with local and/or network information related to a user(s) of UE 602 and the UE 602, and the services and applications provided by cloud system 606 and/or restoration engine 700.

In some embodiments, for example, cloud system 606 can provide a private/proprietary management platform, whereby engine 700, discussed infra, corresponds to the novel functionality system 606 enables, hosts and provides to a network 604 and other devices/platforms operating thereon.

In some embodiments, the exemplary computer-based systems/platforms, the exemplary computer-based devices, and/or the exemplary computer-based components of the present disclosure may be specifically configured to operate in a cloud computing/architecture 606 such as, but not limiting to: infrastructure a service (IaaS), platform as a service (PaaS), and/or software as a service (SaaS) using a web browser, mobile app, thin client, terminal emulator or other endpoint.

According to some embodiments, database 608 may correspond to a data storage for a platform (e.g., a network hosted platform, such as cloud system 606, as discussed supra), a plurality of platforms, and/or UE 602. Database 608 may receive storage instructions/requests from, for example, engine 700 (and associated microservices), which may be in any type of known or to be known format, such as, for example, standard query language (SQL). According to some embodiments, database 608 may correspond to any type of known or to be known storage, for example, a memory or memory stack of a device, a distributed ledger of a distributed network (e.g., blockchain, for example), a look-up table (LUT), and/or any other type of secure data repository.

Restoration engine 700, as discussed above and further below in more detail, can include components for the disclosed functionality. According to some embodiments, restoration engine 700 may be a special purpose machine or processor, and can be hosted by a device on network 604, within cloud system 606, on UE 602. In some embodiments, engine 700 may be hosted by a server and/or set of servers associated with cloud system 606.

According to some embodiments, as discussed in more detail below, restoration engine 700 may be configured to implement and/or control a plurality of services and/or microservices, where each of the plurality of services/microservices are configured to execute a plurality of workflows associated with performing the disclosed dental management. Non-limiting embodiments of such workflows are provided below.

According to some embodiments, as discussed above, restoration engine 700 may function as an application provided by cloud system 606. In some embodiments, engine 700 may function as an application installed on a server(s), network location and/or other type of network resource associated with system 606. In some embodiments, engine 700 may function as an application installed and/or executing on UE 602. In some embodiments, such application may be a web-based application accessed by UE 602 and/or devices over network 604 from cloud system 606. In some embodiments, engine 700 may be configured and/or installed as an augmenting script, program or application (e.g., a plug-in or extension) to another application or program provided by cloud system 606 and/or executing on UE 602.

As illustrated in FIG. 7, according to some embodiments, restoration engine 700 includes identification module 702, analysis module 704, determination module 706 and output module 708. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 700 and each of its modules, and their role within embodiments of the present disclosure will be discussed below.

Turning to FIG. 8, Process 800 provides non-limiting example embodiments of the disclosed framework that provides features, capabilities and/or functionality for enhanced and/or improved dental restoration.

According to some embodiments, as discussed herein and in more detail below, Process 800 provides steps for the disclosed automated ceramic inkjet printing in dental restoration manufacturing. As discussed below, in some embodiments, the disclosed framework for ceramic inkjet dental restorations embodies a novel approach to leveraging advanced technologies for manufacturing precise dental components. For example, the disclosed framework integrates 3D scanning, spatial analysis, and precision inkjet printing capabilities, enabling an efficient and high-quality restoration process.

According to some embodiments, Step 802 of Process 800 can be performed by identification module 702 of restoration engine 700; Step 804 can be performed by analysis module 704; Step 806 can be performed by determination module 706; and Steps 808 and 810 can be performed by output module 708.

According to some embodiments, Process 800 begins with Step 802 where engine 700 can perform spatial recognition and oriental analysis operations. In some embodiments, engine 700 initiates its operation by performing a comprehensive 3D scanning of the dental restoration component. Such operation involves capturing, collecting or otherwise identifying detailed spatial coordinates of a dental restoration, providing a foundational framework for subsequent processing. The scanning process generates a precise 3D model of the restoration piece, overlaying the scanned data with predetermined design specifications. This overlay creates a spatial relationship between the physical object and its intended final form.

In some embodiments, engine 700 can employ multiple reference points within the operational space, ensuring accurate positioning of the restoration piece. By correlating the physical position of the restoration with its corresponding 3D surface, implicit and/or explicit representations of geometry along with associated optical properties (e.g., as provided via a STL (Standard Triangle Language) file, for example) and bitmap data, engine 700 pinpoints application zones for subsequent color and glaze treatments.

In some embodiments, such spatial recognition operations performed by engine 700 can incorporate advanced artificial intelligence and/or machine learning (AI/ML) algorithms to calculate optimal orientation angles for multi-side processing, ensuring that the framework can address the complex geometries often encountered in dental restorations. Such algorithms rely on high-resolution imaging and computational models to analyze the geometry of each piece, accounting for factors like surface curvature and edge contours.

Accordingly, in some embodiments, Step 802 can involve engine 700 implementing any type of known or to be known computational analysis technique, algorithm, mechanism or technology to perform the recognition and orientation analysis.

In some embodiments, engine 700 may include a specific trained AI/ML model, a particular machine learning model architecture, a particular machine learning model type (e.g., convolutional neural network (CNN), recurrent neural network (RNN), autoencoder, support vector machine (SVM), and the like), or any other suitable definition of a machine learning model or any suitable combination thereof.

In some embodiments, engine 700 may be configured to utilize one or more AI/ML techniques chosen from, but not limited to, computer vision, feature vector analysis, decision trees, boosting, support-vector machines, neural networks, nearest neighbor algorithms, Naive Bayes, bagging, random forests, logistic regression, physics-based rendering, differentiable rendering, inverse rendering, neural rendering, and the like. By way of a non-limiting example, engine 700 can implement an XGBoost algorithm for regression and/or classification to analyze the dental and/or patient data, as discussed herein.

In some embodiments and, optionally, in combination of any embodiment described above or below, a neural network technique may be one of, without limitation, feedforward neural network, radial basis function network, recurrent neural network, convolutional network (e.g., U-net) or other suitable network. In some embodiments and, optionally, in combination of any embodiment described above or below, an implementation of Neural Network may be executed as follows:
a. define Neural Network architecture/model,
b. transfer the input data to the neural network model,
c. train the model incrementally,
d. determine the accuracy for a specific number of timesteps,
e. apply the trained model to process the newly-received input data,
f. optionally and in parallel, continue to train the trained model with a predetermined periodicity.

In some embodiments and, optionally, in combination of any embodiment described above or below, the trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the aggregation function may be a mathematical function that combines (e.g., sum, product, and the like) input signals to the node. In some embodiments and, optionally, in combination of any embodiment described above or below, an output of the aggregation function may be used as input to the activation function. In some embodiments and, optionally, in combination of any embodiment described above or below, the bias may be a constant value or function that may be used by the aggregation function and/or the activation function to make the node more or less likely to be activated.

Thus, such AI/ML modes can be utilized to predict and/or adapt variations in dental restoration shapes, enabling engine 700 to process an array of designs with minimal, if any at all, human-in-loop intervention.

In some embodiments, Step 802 can involve engine 700 utilizing laser-based distance measurement tools to confirm the alignment of the restoration piece within the operational space. Such tools ensure that the scanned model's dimensions align with the physical object, minimizing errors in spatial representation.

In Step 804, engine 700 can perform bitmap processing. According to some embodiments, as discussed herein, such bitmap processing can involve, but not be limited to, a preconfigured or predefined bitmap, a retrieved bitmap, stored bitmap, dynamically determined bitmap, fine-tuned bitmap, and the like, or some combination thereof. Thus, as provided below, such bitmap, regardless of the type, includes information related to a configuration for placement of a dental restoration component respective to a printhead of a printer (as discussed above respective to FIGS. 1-5).

Accordingly, in some embodiments, Step 804 can involve engine 700 implementing dynamic bitmap generation capabilities, thereby allowing for a flexible approach to processing. While the current implementations typically involve a standardized four-side plus top processing approach, engine 700 adapts based on the size, geometry, and surface complexity of the restoration. This adaptability ensures efficient and precise processing tailored to each unique case.

In some embodiments engine 700 can utilize, for example, 3D-based algorithms for bitmap generation, with ongoing enhancements to incorporate resident processing capabilities. Such capabilities enable real-time bitmap adjustments informed by immediate scan data, allowing engine 700 to compensate for slight positioning variations without the need for extensive robotic adjustments. Such dynamic adaptability reduces complexity and enhances the efficiency of the overall process.

In some embodiments, Step 804's operation by engine 700 can integrate rendering software that supports multi-threaded processing for higher-speed bitmap generation. Such rendering capability allows engine 700 to create high-resolution bitmaps for intricate surface details such as grooves, fissures, layered textures, and the like. Additionally, engine 700's processing mechanisms can include, but are not limited to, predictive modeling tools that simulate the interaction of inks with the ceramic substrate. Such simulations can guide adjustments to ink droplet size, trajectory, velocity, and the like, thereby optimizing application precision, as discussed infra.

In some embodiments, by way of another non-limiting example embodiment, the bitmap processing performed in Step 804 can involve the retrieval, extraction, upload, download or otherwise identification of a preconfigured bitmap. Such bitmap can include information similar to the dynamically determined bitmap discussed above; however, such information can be, but is not limited to, predefined, determined upon request or identification of a restoration component, based on an analysis of the information from within Step 802, discussed above, and the like.

Therefore, in some embodiments, for example, a restoration component can be identified, for which a bitmap can be determined in a similar manner as above (e.g., analysis of the physical representation of the dental restoration component, for example); however, rather than being a dynamic bitmap, the bitmap can be predetermined/preconfigured such that the bitmap information per surface of the restoration component (e.g., shape, sides, and the like) can be obtained and applied via engine 700. Thus, in such embodiments, engine 200 can involve performing Step 802, then based on a predetermined bitmap, proceeding to Step 806.

In Step 806, engine 700 can determine and/or perform multi-axis positioning and control. In some embodiments, to maintain precise spatial relationships during material application, engine 700 can employ a n-axis (e.g., five-axis) positioning system. Such positioning system ensures optimal orientation of the restoration piece throughout all processing phases. By maintaining a threshold (or minimum or range) throw distance of k millimeters (e.g., five millimeters, for example) between the printhead and the restoration surface, a consistent application quality at or equal to a threshold level can be achieved and/or maintained.

According to some embodiments, such positioning control system operates by handling geometries, such as, for example, molar restorations with intricate occlusal surfaces. By maintaining the threshold throw distance and leveraging both physical adjustments and bitmap modifications, engine 700 ensures uniform application quality. Such dual approach enhances the engine 700's ability to handle orientation variations effectively, providing versatility in addressing a wide range of restoration designs.

In some embodiments, for example, a five-axis system can utilize servo-driven actuators for precise, rapid movements along X, Y, Z, rotational, and tilt axes. Such high level of control allows engine 700 to maintain alignment even when processing irregular or asymmetric shapes. For example, printing system 10, discussed supra, can be equipped with collision detection sensors to prevent misalignments or damage during repositioning. Furthermore, integrated gyroscopic stabilization ensures the restoration piece remains steady during the application of inks and glazes, even at high speeds.

In Step 808, engine 700 can perform and/or cause system 10 to perform material application and process control of the applied slurry to the dental restoration component (e.g., replacement tooth or crown, for example). In some embodiments, engine 700's material application process is a multi-stage procedure utilizing distinct printheads for stain and glaze applications. A non-limiting example embodiment configuration, for example, includes four printheads: three for color components (yellow, blue, and brown) and one for glaze application. Such segregation allows for precise control of material composition and application parameters. It should be understood, however, by those of ordinary skill in the art that variations in the number of printheads and/or types of components would not deviate from the scope of the instant disclosure.

For example, in some embodiments, each printhead can incorporate piezoelectric technology for precision inkjet delivery. Such technology uses voltage pulses to control the ejection of ink droplets, achieving drop-on-demand functionality that ensures consistent application. The ink delivery component(s) is equipped with temperature-regulated nozzles to maintain consistent viscosity and flow characteristics across different material types. Such precision ensures that the stains and glazes adhere uniformly to the ceramic substrate, producing a high-quality finish.

In some embodiments, preheating mechanisms positioned away from the printheads can optimize substrate conditions, ensuring accurate drop placement and adhesion without premature ink drying. Such thermal management strategy enhances drop placement accuracy and adhesion while maintaining optimal printhead operating conditions. Furthermore, engine 700's control over material ratios—such as the current 30% glaze to 70% pigment composition—can be fine-tuned (e.g., via AI/ML analysis and processing, as discussed above) to achieve desired aesthetic and functional outcomes.

In some embodiments, the discussed material application processing can incorporate real-time monitoring of ink flow rates and droplet formation using inline sensors. Such sensors can detect anomalies in, for example, droplet size, thereby ensuring immediate adjustments to maintain consistency. In some embodiments, glaze application processing can employ a variable layer thickness approach, whereby engine 700 can cause dynamic adjustments to the amount of glaze based on the geometry and surface requirements of each restoration piece. This ensures uniform coating while minimizing material waste. Moreover, this provides functionality to impact (e.g., influence, weight and/or modify) micro-texture details which can substantially improve the esthetic mimicry of the restoration, as discussed herein.

In Step 810, engine 700 can perform operations related to quality assurance and validation. According to some embodiments, engine 700 can perform quality assurance operations upon the application of the ceramic slurry, as part of Step 808, and/or upon the performance of each step of the restoration process of Process 800, discussed above. From initial spatial scanning to final material application, engine 700 can enforce stringent control over critical parameters, coupled with real-time monitoring to ensure consistent (and/or threshold satisfying) throw distances, proper material ratios, and accurate validation of surface characteristics.

In some embodiments, such quality assurance mechanisms can extend to thermal management, material application, and final appearance verification. By incorporating such controls, engine 700 delivers reliable results across varying restoration geometries and specifications.

Engine 700 can perform such quality assurance operations via AI/ML models, as discussed above. For example, engine 700 can implement machine vision models for post-process validation. For example, engine 700 can capture high-resolution images of the finished restoration, analyzing them via such vision models for defects such as uneven coating, color mismatches, or glaze inconsistencies. Any identified issues are flagged for corrective action, ensuring that only components meeting the desired specifications are approved. The validation process also includes adherence testing, where the bond strength of the applied materials is evaluated to confirm durability under operational conditions.

According to some embodiments, Step 810 can provide an output or electronic/digital report via control software that advised on key perform indicators (KPIs), such as, but not limited to, material utilization rates, processing times, error occurrences, and the like, or some combination thereof. Such insights enable continuous improvement, ensuring the system remains at the forefront of precision dental restoration manufacturing.

According to some embodiments, engine 200, via the steps of Process 800, can operate to maintain certain optimizations-for example, focusing on reducing cycle times and enhancing overall efficiency. For example, current processing times range from 90 to 120 seconds per restoration. Efforts to streamline these intervals include optimizing robotic movements, refining thermal processes, and improving ink formulations to reduce drying times. Such strategies not only accelerate production but also maintain or enhance the quality of the final product.

Moreover, in some embodiments, layer management is critical in such optimization. For example, for stain applications, engine 700 can utilize high-density pigment formulations to achieve optimal opacity and color density in single-pass applications. Glaze applications, however, may require multiple passes to achieve desired thickness and surface characteristics. Therefore, engine 700 can ensure, via Step 810, proper binding between layers, enhancing both aesthetic appeal and durability.

Accordingly, as discussed above, the processing of the steps of Process 800 via engine 700, improved systems and methods for ceramic inkjet dental restorations is provided, which combines spatial recognition, dynamic processing architecture, multi-axis positioning, material application control, and robust quality assurance mechanisms. The disclosed methodologies and continuous optimization efforts provide a comprehensive computerized technical solution that addresses the challenges of modern dental restoration manufacturing while achieving improved quality and efficiency.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, and the like).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, and the like).

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data. Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A method comprising:
analyzing, by a device, a dental restoration component, and determining information related to a physical representation of the dental restoration component;
generating, by the device, based on the determined information, a dynamic bitmap for the dental restoration component, the bitmap comprising adaptive information related to characteristics of the dental restoration component;
determining, by the device, based on the dynamic bitmap, multi-axis positioning for a printhead, the multi-axis positioning corresponding to a threshold throw distance of the printhead to a surface of the dental restoration component; and
causing, by the device and the printhead, material application to the dental restoration component based on the multi-axis positioning.

2. The method of claim 1, further comprising:
performing three-dimensional (3D) scanning of the dental restoration component;
determining, based on the 3D scanning, spatial coordinates of the dental restoration component; and
performing the determination of the information based on the determined spatial coordinates.

3. The method of claim 2, further comprising:
generating a 3D model of the dental restoration component; and
generating a simulation of the 3D model by overlaying a design specification over the 3D model, wherein information related to the simulation is comprised within the determined information.

4. The method of claim 1, further comprising the characteristics comprising at least one of size, geometry or surface complexity.

5. The method of claim 1, further comprising the multi-axis positing being for a set of printheads associated with the device.

6. The method of claim 1, further comprising the throw distance being a maximum of 5 millimeters.

7. The method of claim 1, further comprising the multi-axis positioning comprising information related to a set of axes selected from a group consisting of: X, Y, Z axes; rotational axis; and a tilt axis.

8. The method of claim 1, further comprising the material application comprising at least one of a stain application or glaze application.

9. The method of claim 1, further comprising:
performing validation of the material application, the validation corresponding to a threshold-based evaluation, the evaluation based on at least one of the throw distance, material ratio or accuracy.

10. The method of claim 1, further comprising the device being an inkjet printer.

11. A device comprising:
a processor configured to:
analyze a dental restoration component, and determine information related to a physical representation of the dental restoration component;
generate, based on the determined information, a dynamic bitmap for the dental restoration component, the bitmap comprising adaptive information related to characteristics of the dental restoration component;
determine, based on the dynamic bitmap, multi-axis positioning for a printhead, the multi-axis positioning corresponding to a threshold throw distance of the printhead to a surface of the dental restoration component; and
cause, via at least the printhead, material application to the dental restoration component based on the multi-axis positioning.

12. The device of claim 11, wherein the processor is further configured to:
perform three-dimensional (3D) scanning of the dental restoration component;
determine, based on the 3D scanning, spatial coordinates of the dental restoration component; and
perform the determination of the information based on the determined spatial coordinates.

13. The device of claim 12, wherein the processor is further configured to:
generate a 3D model of the dental restoration component; and
generate a simulation of the 3D model by overlaying a design specification over the 3D model, wherein information related to the simulation is comprised within the determined information.

14. The device of claim 11, wherein the processor is further configured such that the characteristics comprises at least one of size, geometry or surface complexity.

15. The device of claim 11, wherein the processor is further configured such that the multi-axis positing is for a set of printheads associated with the device.

16. The device of claim 11, wherein the processor is further configured such that the throw distance is a maximum of 5 millimeters.

17. The device of claim 11, wherein the processor is further configured such that the multi-axis positioning comprises information related to a set of axes selected from a group consisting of: X, Y, Z axes; rotational axis; and a tilt axis.

18. The device of claim 11, wherein the processor is further configured such that the material application comprises at least one of a stain application or glaze application.

19. The device of claim 11, wherein the processor is further configured to:
perform validation of the material application, the validation corresponding to a threshold-based evaluation, the evaluation based on at least one of the throw distance, material ratio or accuracy.

20. A method comprising:
identifying, by a device, information related to a physical representation of a dental restoration component;
determining, by the device, based on the identified information, multi-axis positioning for a printhead, the multi-axis positioning corresponding to a threshold throw distance of the printhead to a surface of the dental restoration component; and
causing, by the device and the printhead, material application to the dental restoration component based on the multi-axis positioning.

21. The method of claim 20, further comprising:
analyzing the dental restoration component; and
determining the information related to the physical representation of the dental restoration component.

22. The method of claim 20, further comprising:
generating, based on the identified information, a bitmap for the dental restoration component, the bitmap comprising a configuration for placement of the dental restoration component respective to the printhead; and
performing the determination of the multi-axis positioning for the printhead based further on the bitmap.

23. The method of claim 22, further comprising the bitmap comprising adaptive information related to characteristics of the dental restoration component; and
dynamically applying, by the device, the bitmap based on the adaptive information during the material application.

24. A device comprising:
a processor configured to:
identify information related to a physical representation of a dental restoration component;
determine, based on the identified information, multi-axis positioning for a printhead, the multi-axis positioning corresponding to a threshold throw distance of the printhead to a surface of the dental restoration component; and
cause the printhead to provide a material application to the dental restoration component based on the multi-axis positioning.

25. The device of claim 24, wherein the processor is further configured to:
analyze the dental restoration component; and
determine the information related to the physical representation of the dental restoration component.

26. The device of claim 24, wherein the processor is further configured to:
generate, based on the identified information, a bitmap for the dental restoration component, the bitmap comprising a configuration for placement of the dental restoration component respective to the printhead; and
perform the determination of the multi-axis positioning for the printhead based further on the bitmap.

27. The device of claim 26, wherein the processor is further configured such that the bitmap comprises adaptive information related to characteristics of the dental restoration component; wherein the processor is further configured to dynamically apply the bitmap based on the adaptive information during the material application.
